(19) United States
(12) Patent Application Publication
(10) Pub. No.: US 2007/0005140 A1
(43) Pub. Date: Jan. 4, 2007

(54) Fabrication and use of biocompatible materials for treating and repairing herniated spinal discs

(76) Inventors: Daniel H. Kim, Los Altos, CA (US); Curtis W. Frank, Cupertino, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/359,754

(22) Filed: Feb. 21, 2006

Related U.S. Application Data
(60) Provisional application No. 60/595,394, filed on Jun. 29, 2005.

(51) Int. Cl. A61F 2/44

(52) U.S. Cl. 623/17.16; 623/17.11; 623/902

(57) ABSTRACT

The present invention involves the fabrication and use of biocompatible polymers that are injected percutaneously into the inner portion of a defective region of a spinal disc and swell or expand or subsequently cure in situ to form a disc nucleus prosthesis. The polymers may be synthetic or natural (e.g., collagen), and may be provided in forms including, but not limited to hydrogels, compressible foams, cords, balloons, etc. Subsequent to injection into a target space or void within the disc, one or more cell binding agents, growth factors, and/or drugs on or within the cured polymer then interact with the remaining portion of the disc to support tissue ingrowth and to achieve a higher probability of biological mimicking.

### FIELD OF THE INVENTION

The present invention is related to the minimally invasive repair of intervertebral discs. More particularly, the invention is directed towards the fabrication and use of biocompatible materials to replace at least a portion of the natural intervertebral disc and to support regeneration and restoration of the disc.

### BACKGROUND OF THE INVENTION

The spinal column is formed from a number of bony vertebral bodies separated by intervertebral discs which primarily serve as mechanical cushions between the vertebral bones, permitting controlled motions (flexion, extension, lateral bending and axial rotation) within vertebral segments.

The normal, natural intervertebral disc is comprised of three components:

the nucleus pulposus (“nucleus”), the annulus fibrosis (“annulus”), and two opposing vertebral end plates. The two vertebral end plates are each composed of thin cartilage overlying a thin layer of hard, cortical bone that attaches to the spongy, richly vascular, cancellous bone of the vertebral body. The nucleus is constituted of a gel-like substance having a high (about 80-85%) water content, with the remainder made up mostly of proteoglycan, type II collagen fibers, and elastin fibers. The proteoglycan functions to trap and hold the water, which is what gives the nucleus its strength and resiliency. The annulus is an outer fibrous ring of collagen fibers that surrounds the nucleus and binds together adjacent vertebrae.

With aging and continued stressing, the nucleus may become dehydrated and may degenerate, and/or one or more rents or fissures may form in the annulus of the disc. Degeneration of the nucleus results in changes in the proportion and types of proteoglycans and collagens (which makes the nucleus more eosinophilic), and a reduction in the total number of lacunae containing viable chondrocytes. In addition, the matrix of the nucleus may break down, with the formation of permeative slit-like spaces. Often there is also disruption of the collagen fiber arrays in the annulus, traumatic damage to the end plate(s), and vessel and nerve growth in the inner annulus and nucleus. Alterations in the function of local cells are causally implicated in these events. Freemont et al. 2002 J Pathol 196, 374-379.

Such fissures may progress to larger tears that allow the gelatinous material of the nucleus to migrate into the outer aspects of the annulus, which may cause a localized bulge or herniation. The herniation puts pressure on the adjacent nerves and/or a portion of the spinal cord. In the event of annulus 6 rupture, as illustrated in FIG. 1A, the nuclear material 4 may escape from the confines of the disc 2, causing chemical irritation and inflammation of the nerve roots.

Posterior protrusions of intervertebral discs are particularly problematic since the nerve roots are posteriorly positioned relative to the intervertebral discs. Impingement or irritation of the nerve roots not only results in pain in the region of the back adjacent the disc, but may also cause radicular pain such as sciatica. Nerve compression and inflammation may also lead to numbness, weakness, and in late stages, paralysis and muscle atrophy, and/or bladder and bowel incontinence.

The most common treatment for a disc protrusion or herniation is discectomy. This procedure involves removal of the protruding portion of the nucleus and, most often, the annular defect does not get repaired, as illustrated in FIG. 1B. Discectomy procedures have an inherent risk since the portion of the disc to be removed is immediately adjacent the nerve root and any damage to the nerve root is clearly undesirable. Further, the long-term success of discectomy procedures is not always certain due to the loss of nucleus pulposus which can lead to a loss in disc height. Loss of disc height increases loading on the facet joints, which can result in deterioration of the joint and lead to osteoarthritis and ultimately to foraminal stenosis, pinching the nerve root. Loss of disc height also increases the load on the annulus as well. As the annulus fibrosis has been shown to have limited healing capacity subsequent to discectomy, a compromised annulus may lead to accelerated disc degeneration, which may require spinal interbody fusion or total disc replacement.

If disc degeneration has not yet resulted in excessive herniation or rupture of the annulus, it may be desirable to perform a nucleus replacement procedure in which the degenerated nucleus is supplemented or augmented with a prosthesis while leaving the annulus intact. Ongoing research in prosthetic nucleus replacement devices includes the utilization of materials such as metal, nonmetal, ceramic, and elastic coils. However, these devices would still require an invasive procedure for implant insertion, which would be accompanied by the associated risks of annular trauma during the implantation. In addition, there may be difficulty in matching the implant size and shape with the disc space.

Accordingly, there is a need for prosthetic implant materials that can be appropriately sized and shaped and delivered to a target site within a vertebral disc in a minimally invasive manner, and that can supplement the existing annulus and/or nucleus pulposus in a process of disc regeneration and restoration.

### SUMMARY OF THE INVENTION

The present invention involves the fabrication and use of biocompatible materials (synthetic, natural, or a combination of both) to replace at least a portion of the natural intervertebral disc. The implantable materials preferably are operative in three stages, which can have different functions and modes of action. The first stage facilitates percutaneous delivery into the intervertebral disc; the second stage provides mechanical and material properties that mimic substantially those of the natural disc or portion thereof that it is replacing; and the third stage enables drug delivery to and regeneration of cells within the remaining portion of the disc. After implantation into the disc void in the first stage, the material is transitioned into its second stage. The second stage includes filling the disc void, and also includes creating an environment that acts as a load-bearing frame structure while being conducive to promoting disc cell regeneration and tissue ingrowth by providing mechanical and material properties that mimic closely those of the natural disc or portion thereof that the material is replacing. In the third stage, one or more cell binding agents, growth factors, and/or drugs interact with the remaining portion of the disc to support tissue ingrowth and to achieve a probability of biological mimicking higher than that achieved by the second stage.

The nucleus of a herniated spinal disc is extruded and is displaced from its normal position within the boundaries of its outer fibrous tissue, the annulus. Herniation puts pressure on a portion of the spinal cord and on the corresponding nerves and results in considerable pain. In an embodiment of the present invention, a biocompatible material is injected percutaneously into the defective region and acts as a substitute for the extruded nucleus, so as to prevent further degeneration of the nucleus.

The subject materials comprise, at least in part, one or more polymers. In certain embodiments, the polymer is in the form of a fluid, a hydrogel, a viscous suspension, a plurality of very small particles, etc., having an initial flowable form to facilitate delivery thereof through percutaneous means. Subsequent to delivery, the material is transitioned (actively or passively) to provide a more rigid and/or solid monolithic form that provides mechanical and material properties that mimic closely those of the natural disc or portion thereof that it is replacing.

In other embodiments, a polymer material is a compressible and/or expandable solid, e.g., foams, cords, balloons. If compressible, the material is provided in a compressed, constricted or constrained state to facilitate percutaneous delivery, such as through a small gauge tube or cannula, into the disc space. Upon release from the tube, the material is expanded, e.g., such as by release from the delivery device, by degradation over time of a biodegradable casing covering the material, or by fluids within the disc system.

The present invention is particularly suitable for replacing a portion of the intervertebral disc nucleus. In comparison to total disc replacement, an injectable disc nucleus has numerous advantages. Since only the nucleus is being replaced, the procedure is considerably less invasive, easier to approach and perform, and easier to revise in the event that additional surgery becomes necessary. The risk of permanent nerve injury is lower and no fixation components are required since the implant is not designed to be affixed to the vertebrae. Further, by replacing only the nucleus, this treatment method could potentially enable the reestablishment of the biomechanical properties of the diseased or degenerative disc while preserving the functions of the remaining disc tissues (i.e., the disc annulus and vertebral endplates). This is desirable for numerous reasons, most notably in preventing or greatly postponing the disc degeneration process that generally occurs from traditional surgical methods. Other advantages include the maintenance of range of motion and mechanical characteristics, restoration of natural disc height and spinal alignment, and significant pain reduction.

The use of polymers as the implant material is advantageous over other contemplated materials for various reasons. Because the polymers are at least initially in a flowable or conformable state, they can fill any void of any size and shape. In turn, because the entirety of a void may be filled, the stresses on the implant are ideally distributed resulting in a more stable disc. The implants may be further designed to have mechanical properties of a natural disc nucleus, including sharing a substantial portion of the disc's compressive load and restoring the normal load distribution while avoiding excessive wear on the endplate-implant interface.

The implant materials (whether used as a filler material and/or a casing material which contains or covers the filler material) of the present invention may include one or more polymers in any of the below-described forms (e.g., hydrogels, microgel particles, foam, cords, etc.) or may be a polymer precursor (e.g., monomers, oligomers) which, upon reacting with polymerization initiators or crosslinkers, form a polymer. These implantable materials or equivalents thereof may be configured to have any material and/or mechanical properties to restore the disc anatomy and function to its original state or as close to its original state as possible. For example, implants could be a mixture of biodegradable and non-biodegradable materials. More specifically, over time the biodegradable material could accelerate the encapsulation of appropriate cell lines that produce extracellular matrix proteins such as collagen, while the non-biodegradable material would support mechanical loading of the disc until the ingrowth of tissue was sufficient to maintain the integrity of the disc. To this end, the porosity of the implant can be selected to time the biodegradation process accordingly as well as to facilitate the biological functions of the nucleus, including but not limited to fluid diffusion, nutrients transport, and metabolite removal through the disc.

Further, the surface of the implant materials may have modifications to facilitate adhesion between discrete units of implanted material or between the implants and the surrounding tissues or to provide abrasion resistance. The surface may be activated to introduce functional groups thereon. The functional groups may themselves be linked to molecules that are capable of interacting with biological systems or that are capable of being crosslinked in the presence of chemical crosslinking agents. The surface may also be chemically treated, such that the material may be chemically and covalently linked to an additional material, which coats the surface.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1A illustrates a top view of a herniated spinal disc. FIG. 1B illustrates the disc of FIG. 1A upon surgical removal of the herniated portion of the disc.

FIGS. 2A and 2B illustrate the injection of a hydrogel material of the present invention into a void within a spinal disc and the subsequent curing of the material

FIGS. 3A and 3B illustrate the injection of foam units of the present invention into a void within a spinal disc space and the subsequent expansion of the units and the filling of the void.

FIGS. 4A-4E illustrate various steps involved in the fabrication and implantation of encased foam units of the present invention within a disc void.

FIGS. 5A-5E illustrate various steps involved in the implantation of another implant embodiment of the present invention within a disc void, where the embodiment includes a primary and a secondary implant material.

FIGS. 6A and 6B illustrate the injection of a polymer cord into a void within a spinal disc space and the subsequent expansion of the cord and the filling of the void.

FIGS. 7A-7C illustrate an exemplary delivery tool usable to percutaneously inject the implants of the present invention into an intervertebral disc.

FIGS. 8A-8D illustrate the use of various annulus closure mechanisms for use with the present invention.

FIGS. 9A and 9B illustrate open-surface and sandwich structure polyurethane foams, respectively.

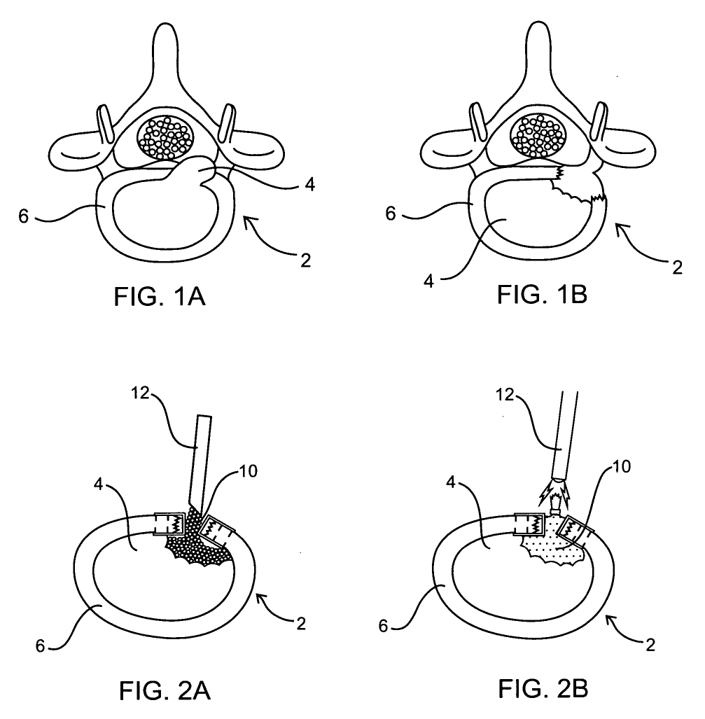

### DETAILED DESCRIPTION OF THE INVENTION

The invention is now described in greater detail, including a description of the types of polymers which are suitable to achieve certain of the objectives of the present invention, the physical and material configurations of the polymers for use as intervertebral disc implants/prostheses, the tools and methods for implanting these materials into intervertebral discs, and the regeneration of disc cells by implanting these materials.

The implantable materials preferably are operative in three stages. The first stage is initial repair. A suitable material that acts as a temporary replacement for the extruded nucleus is implanted at a target site within a vertebral disc. The second stage is filling the disc void in the nucleus pulposus caused by the herniation and also is the creation of an environment that acts as a load-bearing frame structure while promoting disc cell regeneration and tissue ingrowth by providing porosity as well as the mechanical and material properties that mimic closely those of the natural disc or portion thereof that the material is replacing. The third stage is the sustained release of one or more cell binding agents, growth factors, and/or drugs that interact with the remaining portion of the disc to enhance further the mimicking of the natural disc and to promote the repair process of the annulus and nucleus pulposus, so as to restore the original properties of the intervertebral disc.

As used herein, the phrases “mimic closely” and “mimic substantially,” when used in connection with the mechanical and material properties of a natural disc or portion thereof, means approximately the mechanical and material properties of an unherniated natural disc.

As used herein, the phrase “mechanical and material properties” refers to properties such as tensile strength, range of loading forces on the discs at different body positions, range of pressure on the discs at different body positions, the compressive modulus of the disc, and the stiffness coefficients of the disc during axial rotation, anterior compressive shear, posterior compressive shear, and axial compression. Numerical values for these properties are known in the art. See, for example, Nachemson A. Clin Orthop 1966, 45:107-22, which is hereby incorporated by reference.

The nucleus pulposus is under very high pressure when a human is upright. It has two main functions: to bear or carry the downward weight (axial load) of the body, and to act as a pivot point from which all movement of the lower trunk occurs. A third function of the nucleus pulposus is to act as a ligament and to bind the vertebrae together.

Polymers

Small molecules (monomers) can be combined to form larger molecules (polymers) through a process called polymerization. There are two types of polymerization processes: condensation (step-growth) polymerization and addition (chain-growth) polymerization.

In a condensation (or step-growth) reaction, a chemical group on one small molecule reacts with a chemical group on a second small molecule in such a way that the two small molecules are connected together into a larger molecule and water is “condensed” out. If each of the starting molecules has at least two reactive chemical groups, the condensation reaction can continue, eventually forming high molecular weight polymers. For example, in the expression below there are two molecules having core chemical structures X and Y (which can represent many different combinations of atoms) as well as two reactive groups, —OH and —COOH, where the first reactive group is an alcohol and the second is a carboxylic acid:
HO—X—OH+HOOC—Y—COOH→HO—X—OOC—Y—COOH+H₂O.
This reaction may proceed further to form a high molecular weight polymer because the product still has two reactive chemical groups (an alcohol and a carboxylic acid). Thus, polymerization of a condensation polymer consists of a series of condensation steps.

Addition (or chain-growth) polymerization involves a chain reaction in which a highly reactive species, such as a free radical, is prepared by decomposition of an initiator molecule. This free radical is highly reactive and will react with a vinyl group that contains a double bond. One example of a monomer of this class is vinyl chloride, which has the structure

If an initiator molecule (referred to as M₂) decomposes to form two free radicals (M′), one of these free radicals can react with a vinyl chloride monomer to form the species

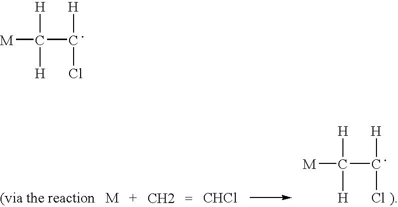

This species then can rapidly react with a series of additional vinyl chloride monomers, much like stringing beads on a necklace, to form a long polymer, polyvinyl chloride (PVC), hundreds or even thousands of monomer units long, as shown below: 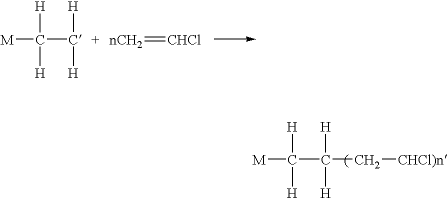
Eventually, the chain reaction stops because the free radical at the end of the chain is lost by one of several possible processes.

The injectable polymers of the present invention are biocompatible, are mechanically strong and sufficiently stable to withstand the natural loads and fatigue undergone by a disc, and are able to achieve polymerization in a reasonable period of time. Furthermore, the polymer should not result in any leakage from the incision or other existing defects. Suitable injectable forms of the polymers include but are not limited to hydrogels, polyurethanes, polymer foams (such as polyurethane), polymer cords (such as polyurethane), microgels and balloons. Several such injectable polymers are described below in greater detail.

Cured polymer foams may be compacted into a delivery tool or device and injected into the defective region where they expand to fill the void caused by the herniation. Single or multiple units of polymer foams may be employed. Multiple units may be affixed to each other, and potentially to the surrounding tissue, by surface modification of the foam with functional groups or by an additional injection of tissue glue. In one embodiment, the functional groups are activated with ultraviolet or visible radiation, e.g., via fiber optic illumination. The thus-delivered final polymer foams will have the proper porosity for tissue ingrowth.

Polymer cords may be composed of multiple fibers of the same or different synthetic and/or natural polymers. Polymer cords may composed of a fully interpenetrating network of a hydrogel or a rubber-like polymer. Multiple units may be affixed to each other, and potentially to the surrounding tissue, by surface chemical modification of the cord with functional groups or by an additional injection of tissue glue. The thus-delivered final polymer cords will have the proper porosity for tissue ingrowth.

Biodegradable polymers are advantageous over nondegradable polymers. Biodegradable polymers permit the ultimate restoration of tissue architecture without the presence of foreign material, have the potential for the controlled delivery of therapeutic agents as the polymer degrades, and incur little or no risk of delayed immune response or rejection. Tissue regeneration requires space, which is provided as the polymers degrade. To be biodegradable, the polymer scaffolds must be able to be hydrolyzed or degraded enzymatically into products that can be metabolized or excreted from the body.

A preferred biodegradable polymer for use in the present invention is one that may be shaped as a film and/or shaped as a vacuum bag, has enough strength to hold a compressed polymer foam, and has a short degradation time. Suitable biodegradable polymers include poly (d,1-lactic co-glycolic acid), poly (1-lactic acid), alginates, and various hydrogels.

The rate of scaffold degradation can be controlled, and should be tailored to allow cells to proliferate and secrete their own extra cellular matrix while the polymer scaffold gradually disappears over a desired period ranging from days to months to leave enough space for new tissue growth. Because the mechanical strength of a scaffold usually decreases with degradation time, the degradation rate may be required to match the rate of tissue regeneration in order to maintain the structural integrity of the implant. The rate of tissue regeneration is itself dependent on the presence of cell binding agents, growth factors, and small drug molecules. The various factors that may affect the degradation rates of polymer scaffolds are summarized in Table 1.

TABLE 1

Polymer Chemistry
Composition
Structure
Configuration
Morphologic features
Molecular weight
Molecular weight distribution
Chain motility
Molecular orientation
Surface to volume ratio
Ionic groups
Impurities or additives
Scaffold Structure
Density
Shape
Size
Mass
Surface texture
Porosity
Pore size
Pore structure
Wettability
Processing method and conditions
Sterilization
In Vitro Conditions
Degradative medium
pH
Ionic strength
Temperature
Mechanical loading
Type and density of cultured cells
In Vivo Conditions
Implantation site
Access to vasculature
Mechanical loading
Tissue growth
Metabolism of degradation products
Enzymes

From Lu 2001 Clin Orthop 391, S251-S270

Hydrogels are cross-linked polymeric structures containing either covalent bonds produced by the simple reaction of one or more comonomers, physical cross-links from entanglements, association bonds such as hydrogen bonds or strong van der Waals interactions between chains, or crystallites of two or more macromolecular chains. Hydrogels swell in water, but do not dissolve. There are many different macromolecular structures that are possible for physical and chemical hydrogels. In addition to their hydrophilic character and potential for biocompatibility, hydrogels are chemically stable and may be configured to degrade and eventually disintegrate and dissolve. Hydrogels are used widely as biomaterials because of their hydrophilicity, biocompatibility, and other advantageous physical properties. They are capable of encapsulating proteins or mammalian cells for applications such as biosensors, cell transplantation, and drug delivery.

Polymeric materials that are suitable for use as hydrogels include polyethylene glycol (PEG), polyacrylamide, and ethylene-acrylic acid copolymers. PEG is commonly used in tissue engineering owing to its biocompatibility. PEG is nontoxic, non-immunogenic, non-antigenic, and highly soluble in water. PEG is characteriuzed as a hydrophilic polymer that can be crosslinked by modifying each end of the polymer with either acrylates or methacrylates.

In order to enhance the mechanical properties of hydrogels, they may be provided in an interpenetrating polymer network (IPN) or a double network system (DNS). An IPN is any material containing at least two polymers, each in network form. The three conditions for eligibility as an IPN are: (1) the two polymers are synthesized and/or crosslinked in the presence of the other, (2) the two polymers have similar kinetics, and (3) the two polymers are not dramatically phase separated. However, polymers which are synthesized separately to form only a single crosslink and those polymers which have vastly different kinetics are still considered to be IPNs. Both the tensile and compressive strengths of a hyrdogel can be significantly increased over a single IPN.

DNSs were developed in order to overcome the lack of mechanical strength of hydrogels. A DNS is a two-component interpenetrating network with high mechanical strength and high water content. DNSs have been reported to possess 20 to 50 times the enhanced mechanical properties of ordinary hydrogels. A DNS may be synthesized, for example, by modifying PEG with an acrylate to form a first crosslinked network of PEG-diacrylate. The first network is then treated with ethylene glycol dimethacrylamide (EGDMA), which is an additional crosslinking agent, and acrylamide monomer to produce a double network hydrogel of PEG-acrylamide.

Hydrogel compositions may be customized to provide the desired characteristics for a particular application. For example, pH-sensitive hydrogels have the ability to respond to pH changes. In order to have pH-sensitivity, the gels need to contain ionizable side groups such as carboxylic acid or amine groups. In acidic media, the gels do not swell so much, but in neutral or basic media, the gels swell significantly due to ionization of the pendant acid group. Another type of hydrogel, temperature-sensitive hydrogels, swell within a selected temperature range. (See Hirotsu, S.; Hirokawa, Y.; Tanaka, 1987 T. J Chem. Phys 87, 1392, in which the LCST of a synthesized cross-linked poly(N-isopropyl acrylamide) (PNIPAAm) was determined to be 34.3° C.).

In one aspect of the present invention, hyrdogels are provided in the form of very small particles (“microgel” particles). Preferably, the microgels are comprised of a fully interpenetrating network of at least two hydrogel materials. The microgel particles may be made of normal, pH-sensitive or temperature-sensitive hydrogel that would swell upon contact within the body. Microgels may be prepared by emulsifying an aqueous solution of PEG and a non-aqueous medium, such as silicone oil of an appropriate viscosity. The microgels are cured by treating the silicone oil emulsion with UV light. Fully cured microgels are washed to remove all traces of any residual monomers and silicone oil as well as any photoinitiators, crosslinking agents, and/or surface coupling agents that may have been used in the curing process.

The microgel particles may have a diameter in the range from about 10 μm to about 500 μm where the particles have the same size or varying sizes in order to effect the desired porosity to optimize tissue ingrowth. The size of the microgels can be varied by changing the viscosity of the silicone oil during emulsification. Different porosities may be achieved by a substituting a portion of microgels having a given diameter with microgels of a different diameter. All or some of the microgel particles may be provided affixed to each other prior to implant or may be designed to do so subsequent to implant. They may be further designed to affix themselves to the surrounding tissue in order to secure the microgel particles in place. This may be achieved through surface chemical modification of the cured microgel particles with functional groups that could be activated with ultraviolet or visible radiation via fiber optic illumination. Tissue glue could also be additionally applied to the defective region.

Injection and curing of the hydrogel/microgel particles may be performed in any suitable percutaneous manner including those disclosed in U.S. patent application Ser. No. 11/120,639 filed on May 2, 2005, herein incorporated by reference in its entirety. Typically, microgels are injected as a paste. An example of an injection process is illustrated in FIG. 2A in which microgel particles or beads 10 are delivered in a selected amount or volume through a needle or small gauge cannula 12, through the annulus 6 into the nucleus 4 of a disc 2. Subsequent to delivery within the disc void(s), the microgel particles 10 may remain in the same form or take another form either by curing by the application of heat or UV light, as illustrated in FIG. 2B, or by absorption of surrounding fluids, i.e., where the prosthesis material is hydrophilic, or by the application of another substance or chemical which reacts with the material in a way that changes its form.

The surface of microgels and hydrogels may be modified chemically in order to facilitate bonding with other microgels so as to form one hydrogel unity and in order to facilitate adhesion to cells. Moieties such as N-hydroxysuccinimide (NHS) groups or azide groups may be added to the surface. The NHS groups react with the acrylamide comonomer in the second network of the DNS, which permits bonding of the microgel particles when treated with UV light. The azide groups react with any carbon-hydrogen bond, which permits bonding between microgels and surrounding tissue. If an azide modified polymer is incubated with collagen, collagen bonds to the azide groups on the surface of a polymer. The collagen bonding improves spreading and cell adhesion.

In yet another aspect of the invention, the injectable material is a foam. Foams are comprised of a microcellular structure, produced by gas bubbles formed during the polyurethane polymerization mixture. Similar to a coiled metal spring, the flexible foam shows relatively low load-bearing properties with high recovery properties while the rigid foam displays high load-bearing, but with a definite yield point and subsequent cellular collapse and lack of recovery. Preferably the foam has a porosity which facilitates tissue ingrowth. These foams may be made in large blocks in either a continuous-extrusion process, or in a batch-process. Alternatively, they may be individually molded into discrete components or units having particular shapes and sizes, which may be identical or vary from unit to unit. Foams are advantageous in that they provide useful structural properties, porosity that facilitates tissue ingrowth, consistency in size, consistency in pore size, and the ability to be shaped into various forms.

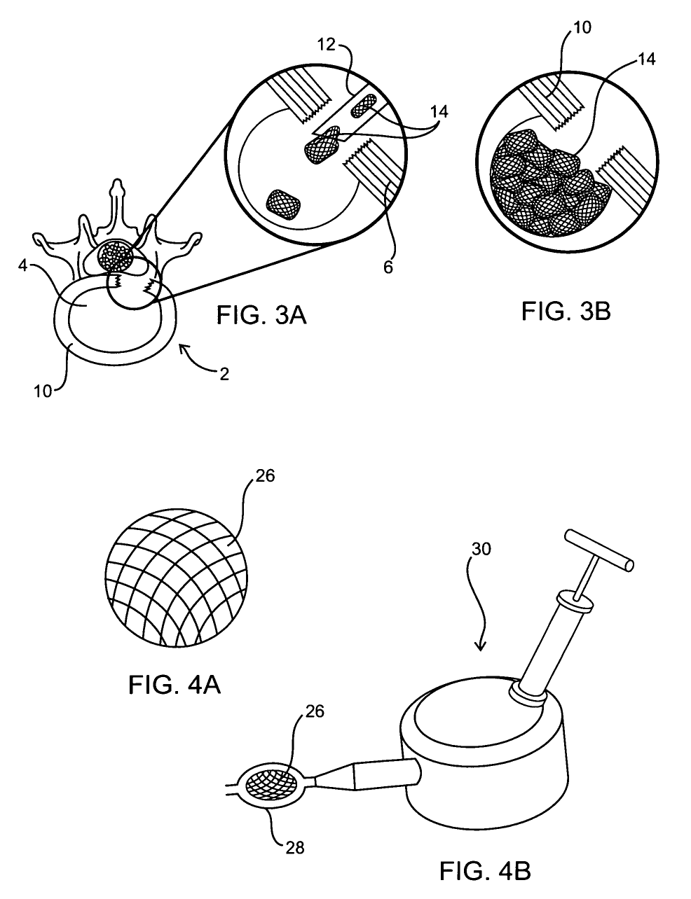

In the context of the present invention, fully cured polymer foam, in the form of either single or multiple units 14, are compressed and compacted into a delivery tool 12 and injected into the defective region or void of the disc space, as illustrated in FIG. 3A. Upon injection into the nucleus, i.e., expulsion from a delivery tool, the foam unit(s) 14 expands to fill and conform to the disc void, as illustrated in FIG. 3B.

The filling of the disc void is based on a precise calculation, which results in a controlled expansion wherein the foam expands only enough to encompass the disc defect. The foam should not overexpand beyond the disc defect and/or overexpand so as encapsulate material within the nucleus. It is generally accepted that the adsorption of plasma proteins onto an artificial surface is the first event to occur when blood contacts a biomaterial, usually within a few seconds, preceded only by the adsorption of water and inorganic ions. Adsorption is not a static event, as adsorbed proteins can undergo conformational changes with time and exchange with other molecules in the contacting solution. It also is accepted that the adsorbed protein layer influences the nature of subsequent events, as other blood components, such as blood cells, must interact with this protein layer.

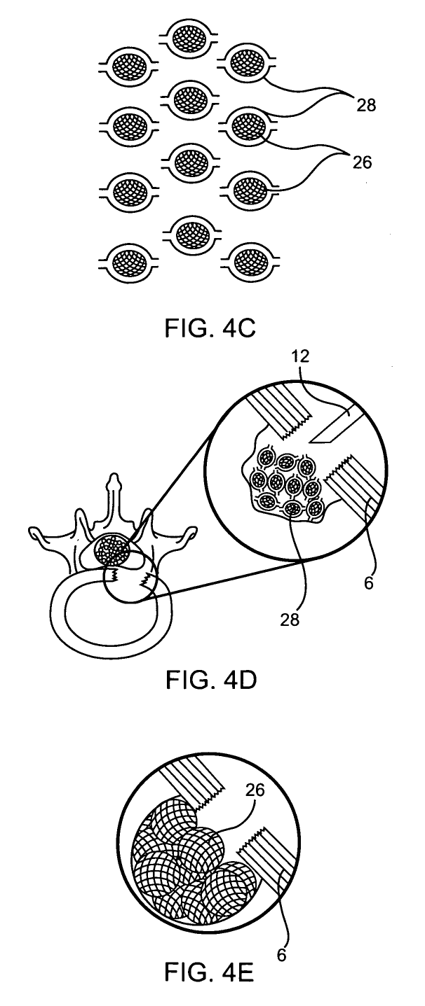

Alternatively, as illustrated in FIGS. 4A-4E, fully cured foam units 26 may be compressed and encased within biodegradable vacuum bags or casings 28, such as by use of a vacuum pump 30 (shown in FIG. 4B). A plurality of the encased units is then injected into the target disc region, i.e., by way of a delivery tool, as illustrated in FIG. 4D. As the casings 28 degrade, as shown in FIG. 4E, the foam units 26 are restored to their expanded volume.

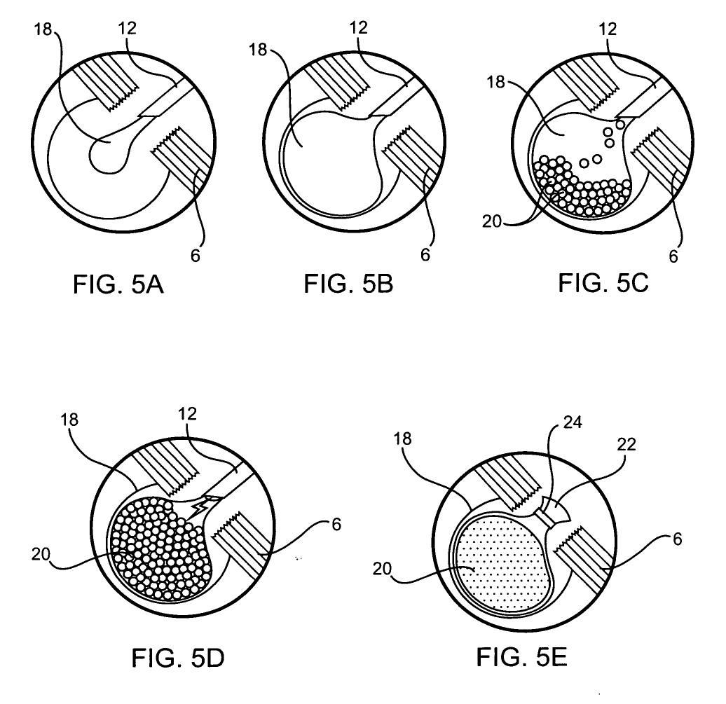

FIGS. 5A-5E illustrate another variation of the invention in which a balloon device 18 is used as a primary implant within a disc void, and in turn, the balloon 18 is filled with a secondary implant material 20, such as microgel particles. First, the balloon device 18 is delivered to within the void, as illustrated in FIG. 5A, and then inflated, as illustrated in FIG. 5B. The inflated balloon 18 is then filled with a flowable filler secondary material 20, as illustrated in FIG. 5C. Alternatively, the flowable filler material 20 could be used initially to expand the balloon thereby eliminating the need for a means of inflation. The filler material 20 may then be cured by exposure to energy, such as ultraviolet or visible radiation via fiber optic illumination, as illustrated in FIG. 5D. Alternatively, polymerization and/or curing could be effected by chemical treatment or exposure to body temperature or absorption of fluid. Optionally, as illustrated in FIG. 5E, the open end 22 of the balloon device 18 may be closed with a clip 24 or by the application of heat. Balloon 18 may be made of a cured polymer material having the same or similar composition and/or properties (e.g., biocompatibility, biodegradability, etc.) as the filler material 20. Further, the balloon material is adapted to be conformable to the walls of the void when in an expanded condition.

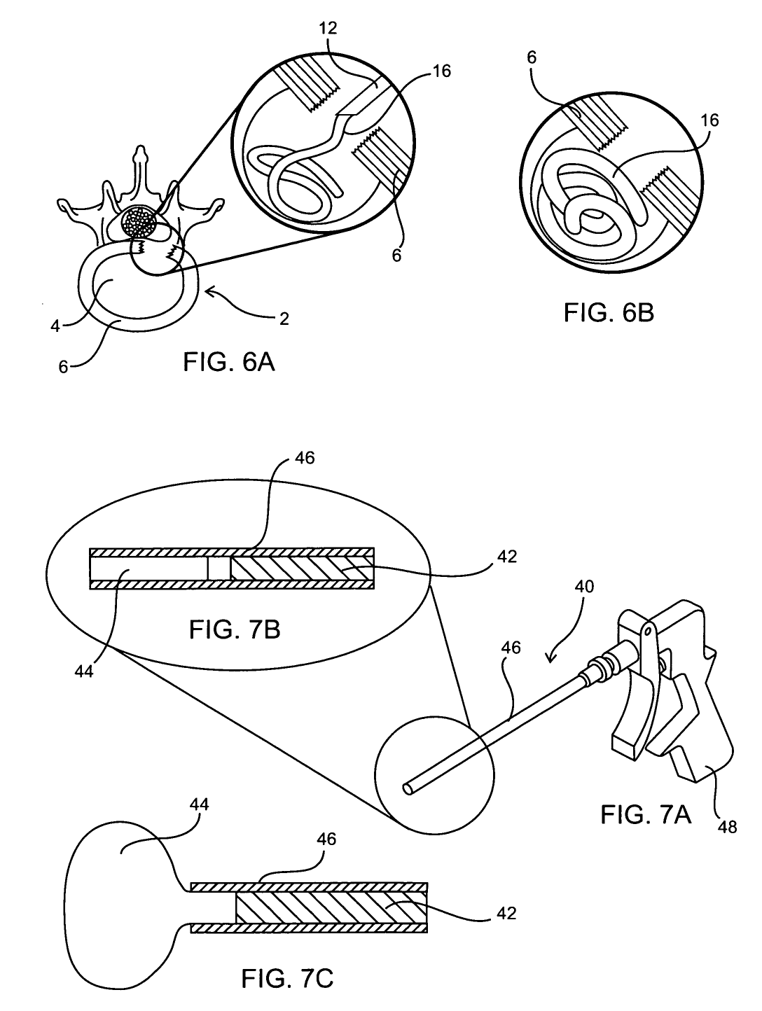

As illustrated in FIGS. 6A and 6B, the injectable material may also be provided in the form of one or more cords 16 composed of multiple polymer fibers. The polymer fibers may be made of hydrogel configured to swell upon contact with the body. Alternatively, the cords may be made of a rubber-like polymer. Similar to the foam, the polymer cord 16 may be compacted or coiled within a delivery tool 12. Upon injection into a disc void, the cord 16 is allowed to expand to fill the area within the cavity. Alternatively, a compressed or coiled cord may be wrapped with a biodegradable casing which degrades upon implantation within the body.

FIG. 7A illustrates a tool 40 for percutaneously delivering and injecting an implant material 44, such as polyurethane in the form of a compressed foam or expandable cord, to an implant site within the body. Tool 40 includes a small gauge or diameter shaft or tube 46 and a handle mechanism 48 at a proximal end of the shaft for advancing a pusher 42 through the shaft's lumen. As shown in FIG. 7B, the implant material 44 is preloaded within the distal end of shaft 46 and in front of pusher 42. Upon positioning the distal end of shaft 46 at the implant site, pusher 42 is distally advanced by actuation of handle mechanism 48, thereby pushing the implant material 44 into the implant site. Upon advancement beyond the distal end of shaft 46, as illustrated in FIG. 7C, the implant material 44 expands to fill the void into which it is delivered. The expansion may be immediate upon implantation where the implant material, such as a compressible foam, is loaded into tool 40 in a compressed state (from a naturally expanded condition). Where the implant material is in the form of a cord in its original or natural state, expansion may occur over a selected period of time after implantation due to the absorption and/or biodegradability of the material.

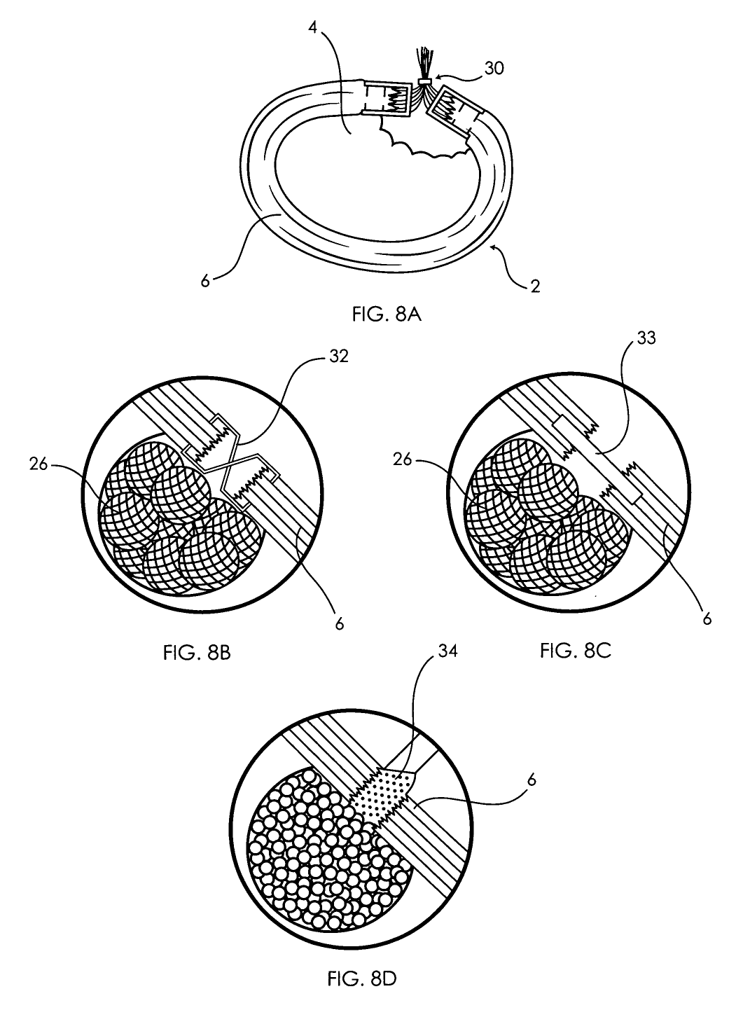

Regardless of the type or form of material implanted, any suitable means may be utilized to seal the opening within the disc annulus. For example, as illustrated in FIGS. 8A, 8B, and 8C, the annulus may be closed by a closure device, such as a clamp, clip, or pin mechanism 30, 32, and 33, respectively, which functions as scaffolding, such as disclosed in U.S. patent application Ser. No. 11/120,639, mentioned above, for closing the annular opening as well as for entraping the nucleus replacment material within the disc space. Alternatively, as illustrated in FIG. 8D, a biocompatible glue 34 may be applied or injected into the opening thereby sealing it.

The closure devices augment the intervertebral disc, including the annulus and or the nucleus, and facilitate repairing and treating as well as preventing degeneration and/or herniation of the intervertebral disc. One or more closure devices are implanted within the disc, most typically within the annulus or within a sub-annular space, or within a void in the annulus, but not necessarily within the annulus itself. The closure materials provide (1) structural support; (2) repairing of the annulus and/or the nucleus; and (3) disc function/mechanism support.

Preferably, the closure materials may be surface treated with cell adhesion molecules or anti-cell adhesion molecules so as to support regeneration of the annulus or the nucleus and/or to mimic the natural biological conditions of the spinal discs, and also to reduce any unnatural environmental changes owing to the addition of the closure materials. Suitable adhesion molecules include RGD, growth factors, collagen, and interleukins. Suitable anti-adhesion molecules include heparin, lectin, and anti-inflammatory compounds. Cell attachment to the side of the closure device that is distal to the nucleus may be minimized by surface treatment with anti-adhesion molecules.

The closure materials may be made of medical implant metals and alloys known to the art. Suitable materials include the various types of titanium known for cell proliferation, cell differentiation, and protein synthesis. See Bächle M et al. 2004 Clin Oral Impl Res 15, 683-692. As set forth above, the closure materials may be surface treated with cell adhesion molecules or anti-adhesion molecules. Surface treatment methods similar those described below for treating the surfaces of polymeric materials may be employed with the closure materials. Surface treatment can enhance cell proliferation, cell differentiation, and protein synthesis of disc-related cell types, such as disc cell and osteoblast cell types.

Optionally, the surfaces of the foam units or cords may be activated or chemically modified with functional groups to enable the units or cords to be affixed to or become affixed to each other and potentially to the surrounding tissue. In the case of a single cord, portions of an intertwined or coiled cord would become affixed to each other. The functional groups could be activated with ultraviolet or visible radiation via fiber optic illumination. For example, the azide group is capable of reacting with any carbon-hydrogen bond, which will permit covalent bonding between units or cords as well as with surrounding tissue. Alternatively or additionally, tissue glue may be injected into the implant site to enhance fixation between the units/cords and between the units/cords and the surrounding tissue.

One example of a suitable polymer for forming the injectable foam or cords of the present invention is polyurethane. A large number of polymers have been used in biomedical applications. Developments in polymer science have produced a variety of synthetic polymers with mechanical properties that resemble biological tissues. Polyurethane elastomers combine excellent mechanical properties with good blood compatibility, which favors their use as biomaterials, particularly as components of implanted devices.

Polyurethanes include those polymers containing a plurality of urethane groups in the molecular backbone, regardless of the chemical composition of the rest of the chain. Thus, a typical polyurethane may contain, in addition to the urethane linkages, aliphatic and aromatic hydrocarbons, esters, amides, urea, and isocyanurate groups. Polyurethanes and the closely related polyureas are the products of the reaction of diisocyanates (—N═C═O) and active hydrogen compounds such as polyols, for example polyglycols, or polyamines as symbolized by the following chemical expression:

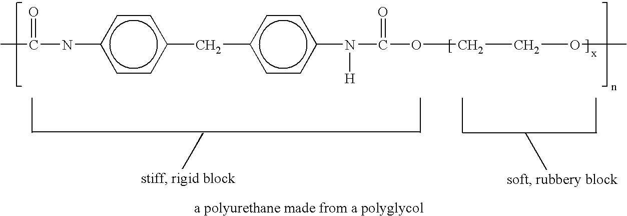

The reaction is catalyzed by mild and strong bases.

Diisocyanates typically employed in polyurethane synthesis include toluene diisocyanate (TDI), methylene bisphenylisocyanate (MDI), hexamethylene diisocyante (HDI), and hydrogenated MDI (HMDI). Isocyanates impart rigidity to the polymer chains; the so-called hard domains or rigid segments are attributable to isocyanates.

A preferred ratio of isocyanate groups to hydroxyl groups is 1.0 to 1.1. If the ratio falls below 1.0, the mechanical strength, hardness, and resilience of the polymer decrease. In addition, elongation and compression set increase sharply.

Polyols are polyfunctional alcohols. Polyols impart high flexibility to the backbone of the network chains; the so-called soft domains or soft segments are attributable to polyols. Low molecular weight polyols produce harder plastics. If the polyols contain three or more hydroxyl groups, crosslinking of the polyurethane occurs. The crosslinked polymer has enhanced mechanical properties relative to the uncrosslinked polymer.

If a blowing agent, such as water, is present during the polymerization process, it will react with isocyanate groups and release a gas, such as CO₂. The released gas creates voids during polymerization, so that the final polymerized product is a foam. Polyurethane foams exist as open-cell or closed-cell reticulated foams. The open-cell foams are highly porous structures because they may contain up to 97% voids. Because of these voids, the foam may be compressed up to 1/15^{th} of its original volume. Open-cell foams allow for the passage of gases, nutrients, and waste products. The foam is easily deliverable, and the expansion size is constant so that only the defmed disc defect or void is encompassed after calculating how much foam is required.

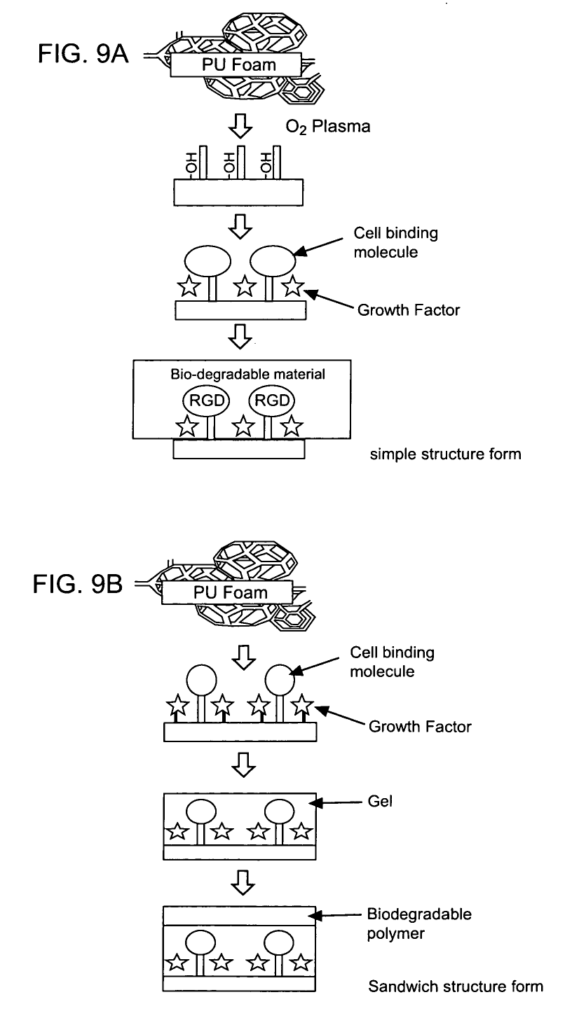

As illustrated in FIG. 9A, the polyurethane foam is activated by treatment with an oxygen plasma that results in formation of hydroxyl groups on the surface of the foam. Cell binding molecules and/or growth factors are bonded to the hydroxyl groups, and the surface is then covered with a biodegradable material. FIG. 9B illustrates a modification of FIG. 9A, wherein the biodegradable material used to cover the surface is itself covered with a biodegradable polymer, so as to form a sandwich structure.

Other polymers that are suitable for use in the present invention include expanded polytetrafluoroethylene (ePTFE), silicone foam, epoxies, polyvinyl chloride (PVC) foam, and poly(d,1-lactic-co-glycolic acid) (PLGA).

Polytetrafluoroethylene (ePTFE) is chemically inert, hydrophobic, and gas permeable. Silicone foam may be made by a process similar to that for making polyurethane foam. If one or more blowing agents are added during polymerization, the resulting foam can be shaped. The foam may also be open-celled. Silicone is biocompatible, and silicone foams have good mechanical properties. Epoxy resins require mixing with a hardener to polymerize and harden. In the presence of hardeners the epoxy groups react and open to produce reactive sites that polymerize. Typical hardeners include m-phenylenediamine and phthalic anhydride. Uncured epoxy resin monomers are toxic.

An epoxy that is biocompatible when cured fully is available from Master Bond, Inc., Hackensack, N.J. 07601. It is a two-component, low-viscosity epoxy resin system designed for bonding, sealing, and potting applications. The USP Class VI-compliant EP21LV system has a non-critical one-to one mix ratio (by weight or volume) and can be cured at ambient or elevated temperatures. Physical strength properties may be adjusted by varying the mix ratio. A mix ratio of two parts resin to one part hardener optimizes strength, rigidity, and hardness, while a mix ratio of one part resin to two parts hardener enhances impact strength, toughness, and flexibility. The cured polymer system demonstrates good adhesion to similar and dissimilar substrates.

Closed-cell PVC foams are one of the most commonly used core materials for the construction of high performance sandwich structures. PVC foams are biocompatible and offer a balanced combination of static and dynamic properties and good resistance to water absorption. PVC foam is marketed in crosslinked and uncrosslinked forms. The uncrosslinked foams, sometimes called linear, are tougher and more flexible.

Over the past few decades, biodegradable polyesters, such as poly(lactic acid) (PLA), poly(glycolic acid) (PGA), and poly(lactic-co-glycolic acid) (PLGA), have been studied extensively for a wide variety of pharmaceutical and biomedical applications. The biodegradable polyester family has been regarded as one of the few synthetic biodegradable polymers with controllable biodegradability, excellent biocompatibility, and high safety. PLGA(poly(lactic-co-glycolic acid)) is an FDA approved polymer which is used in a host of therapeutic devices. It is synthesized by the random co-polymerization of glycolic acid and lactic acid. Successive monomeric units (of glycolic or lactic acid) are linked together in PLGA by ester linkages: With changes in the molar ratio of glycolic acid and lactic acid, the degradation time can be controlled. Exemplary molar ratios include 50/50, 65/35, 85/15, PLGA. Advantageously, PLGA undergoes hydrolysis in the body to produce the original monomers, lactic acid and glycolic acid. These two monomers under normal physiological conditions are by-products of various metabolic pathways in the body. Since the body deals effectively with these two monomers, there is very minimal systemic toxicity associated with using PLGA for drug delivery or biomaterial applications. PLGA has been used for grafts, sutures, implants, and prosthetic devices. PLGA films may be prepared by melting PLGA at about 100° C. or by dissolving in a suitable solvent, such as dichloromethane, tetrahydrofuran, ethyl acetate, chloroform, hexafluoroisopropanol, or acetone, and depositing the melt or solution uniformly on a spin coater. The material hardens into a film after 24 hours.

A non-toxic biodegradable lysine-diisocyanate (LDI)-based urethane has been developed for use in tissue engineering applications. Zhang JY, et al. 2000 Biomaterials 21(12), 1247-58. The polymer matrix was synthesized with highly purified LDI made from the lysine diester. The ethyl ester of LDI was polymerized with glycerol to form a prepolymer. LDI-glycerol prepolymer when reacted with water foamed with the liberation of CO₂ to provide a pliable spongy urethane polymer. The degradation of the LDI-glycerol polymer yielded lysine, ethanol, and glycerol as breakdown products. The degradation products of LDI-glycerol polymer did not affect the pH of the solution significantly.

The physical properties of the polymer network were found to be adequate to support cell growth in vitro, as evidenced by the fact that rabbit bone marrow stromal cells (BMSC) attached to the polymer matrix and remained viable on the surface thereof. Cells grown on LDI-glycerol matrix did not differ phenotypically from cells grown on tissue culture polystyrene plates as assessed by cell growth and by expression of mRNA for collagen type I and transforming growth factor—b1(TGF-b1).

Partially degradable polymers, such as medical grade polyurethane, are also suitable for use in the present invention. These polymers comprise a biodegradable part that promotes disc cell regeneration and tissue ingrowth into the polymer scaffold and a non-degradable part that acts as a load-bearing frame structure. One such suitable structure is a porous, non-degradable polymer in which the pores are filled with a bio-degradable polymer. For example, the cavities of a non-degradable, surface-activated reticulated polyurethane foam may be filed with monomers or prepolymers of a biodegradable polyurethane foam having terminal hydroxyl groups. The surface of the non-degradable polyurethane may be modified to contain free hydroxyl groups if the foam is activated with an oxygen plasma. Plasma treatment is described below. The hydroxyl groups on the non-degradable and degradable foams are reacted with diisocyanate to form new urethane linkages, thereby linking covalently the non-degradable and degradable foams.

Another suitable structure is a biodegradable polymer physically attached to a non-biodegradable polymer. Physical attraction, such as Van der Waals attractions between molecules or hydrogen bonds could hold together two different polymers. For example, a dissolved or melted biodegradable polymer could be glazed onto a non-degradable polymer, resulting in physical bonding of the two polymers.

The time for biodegradation can be calculated, and should be varied depending on the size of the disc defect or void. The calculation requires knowledge of the time required for cell doubling, and therefore knowledge of the number of cells required for regeneration. In another example, the voids of the reticulated polyurethane foam are filled with other biodegradable materials, such as poly(glycolic acid) (PGA), poly(1-lactic acid) (PLA), poly(d,1-lactic-co-glycolic acid) (PLGA), poly(caprolactone), poly(propylene fumarate), poly[1,6-bis (carboxyphenoxy) hexane], tyrosine-derived polycarbonate, ethylglycinate polyphosphazene, and the like. The biodegradable materials promote restoration of the tissue architecture, while the non-degradable scaffold enhances the mechanical properties of the implant by acting as a framework structure. Under the conditions of the human body, the biodegradable parts erode gradually and the remaining foam is a highly porous structure having up to 97% voids. It allows the passage of gasses, nutrients, and waste products, and can sustain mechanical loads. As the degradable polymer degrades, the tissue architecture is restored with the framework structure. The above partially degraded polymer systems are advantageous over biodegradable scaffolds. As bio-degradable scaffolds degrade, the mechanical properties of the regenerated tissues, especially skeletal tissues such as spinal discs, are typically not restored to their original levels.

In addition, biodegradable polyurethane foam surface-treated with cell survival agents/growth factors permits non-competitive slow release of these materials as the foam degrades. Non-competitive slow release into the system generates support of skeletal tissues and/or spinal discs, and enables restoration close to their original state. For example, the cell-binding peptide RGD enhances cell proliferation, cell differentiation enhancement, and cell adhesion. Growth factors will enhance cell survival at the second stage of the implant, and initiate downstream cell-to-cell interaction and cell maintenance.

Activation Treatment Process

In another aspect of the present invention, an activation treatment process may be used to introduce functional groups containing atoms such as oxygen or nitrogen, or to introduce unsaturated bonds onto the surface of the polymeric materials in order to enhance the biocompatibility and/or degradation of the polymeric materials. After surface activation, the introduced functional groups may themselves be reacted, e.g. via graft polymerization, and attached to other materials.

Surface treatment is a fast and efficient method for improving the adhesion properties, abrasion resistance, and other surface characteristics of a variety of polymeric materials. Abrasion resistance is the ability of the surface to withstand abrasion during handling and during implantation according to the method of the present invention. A polymer foam that is abrasion resistant would find use when the foam is delivered in a compressed state by a suitable delivery tool or device.

The extent of the activation treatment is not especially limited; it depends on the purpose of the treatment. Infrared spectroscopy may be employed to monitor the success and extent of the activation treatment. For example, measurement of the absorbance of carbonyl groups before and after the treatment is typically employed as an indication that the activation treatment has been successful. For example, a ratio of the absorbance of carbonyl groups introduced in materials to that from the crystalline region which is not changed by the treatment is estimated by the base line method, which is used to determine the extent of the oxidation by the activation treatment.

For instance, in the case of polypropylene, it is preferable that the ratio of the absorbance at approximately 1710 cm⁻¹attributable to the carbonyl groups introduced in the polymer to the absorbance at approximately 973 cm⁻¹attributable to the methyl groups unchanged in the crystalline region is about 0.2 or less.

The polymeric materials preferably are washed with appropriate solvents to remove impurities before the activation treatment. For example, polyolefins, polyvinyl chloride, and polyvinylidene chloride are preferably washed with an organic solvent, such as methanol or toluene. Cellulose acetate, nylons, polyesters, polystyrene, acrylic resin, polyvinyl acetate, polycarbonate, and polyurethane are preferably washed with an alcohol, such as methanol or ethanol.

Various types of surface treatments may be employed in the context of the present invention, including but not limited to, ozone treatment, ultra-violet light irradiation treatment, high voltage electric discharge treatment, corona discharge treatment, and plasma treatment.

Ozone Treatment

Ozone treatment involves a chemical reaction, namely oxidation of the surface of polymeric materials with ozone molecules upon contact with ozone. The ozone treatment is carried out by exposing the polymeric materials to ozone. Various methods of ozone treatment are available; for example, placing a polymeric material in an ozone atmosphere for a period of time or placing a polymeric material in an ozone stream.

Ozone is produced by passing air, oxygen, or gas containing oxygen such as oxygen-enriched air through an ozone generator. The ozone treatment is carried out by introducing the obtained gas containing ozone into a reaction vessel or a container containing a polymeric material. The conditions of ozone treatment, such as ozone concentration in a gas containing ozone, exposure time, and temperature, will vary with the kind and form of a polymeric material and the nature of the surface activation desired. Typical conditions are an ozone concentration from 0.1 to 200 mg/l, a temperature from 10 to 80° C. and a reaction time from 1 minute to 10 hours. For example, treatment with an ozone concentration from 10 to 40 g/m³ and a time from about 10 to 30 minutes at room temperature is suitable for the treatment of polypropylene and polyvinyl chloride fibers. When the polymeric material is a film, treatment with an ozone concentration of 10 to 80 g/m³ for about 20 minutes to 3 hours is suitable. When air is used instead of oxygen, the ozone concentration becomes about a half of that with oxygen.

Without wishing to be limited to a particular mechanism, it is believed that hydroperoxide groups (—O—OH) are formed, some of which are changed to hydroxide groups and carbonyl groups, on the surface of a polymeric material by treatment, mainly via oxidation, with ozone.

Ultraviolet Radiation Treatment

The surface of polymeric materials may be irradiated with ultraviolet (UV) light. Typically, low-pressure mercury lamps, high-pressure mercury lamps, super high-pressure mercury lamps, xenon lamps, metal halide lamps, and optical fiber systems are employed as UV light sources. Pretreatment of the polymeric material with a solvent before UV radiation increases the absorbance if UV light. Although any wavelength UV light is suitable, a wavelength of 360 nm is preferable in order to decrease the deterioration of the polymeric material. When a polymeric material is irradiated with UV light, a part of the light is absorbed by the chemical structure, such as the double bonds, within the surface of the polymeric material, and some chemical bonds are broken to produce radicals by the absorbed energy. It is believed that the resulting radicals produce carboxylic groups or carbonyl groups via peroxides via reaction with oxygen in the air.

High Voltage Electric Discharge Treatment

With high voltage electric discharge treatment a polymeric material is placed on a conveyor belt roller equipped with a funnel-shaped instrument positioned perpendicular to the belt, and the material is carried by the belt under the narrow end of the funnel. A high voltage such as several thousand volts is sent between a plurality of electrodes attached to the inner wall of the discharge instrument, which creates an electric discharge in the air. The discharge is directed into the wider end of the funnel-shaped instrument and ultimately onto the polymeric material being conveyed below. It is believed that the electric discharge activates the oxygen in air as well as the surface of the material. The activated oxygen is incorporated into the polymeric material and forms polar groups in the polymeric material.

Corona Discharge Treatment

With corona discharge treatment a high voltage of several thousand volts, typically 10 kV, is sent between a plurality of knife-shaped electrodes and a grounded metal roller. The electrodes are attached at intervals of several millimeters to the metal roller. A polymeric material is passed under the electrodes where the corona discharge is generated. This method is especially suitable for films or thin materials.

Plasma Treatment

Both corona discharge and plasma treatment employ electrical ionization of a gas. Plasma (glow) discharge creates a smooth, undifferentiated cloud of ionized gas with no visible electrical filaments. Unlike corona discharge, plasma is created at much lower voltages and temperatures. For the treatment of polymeric material, a cold gas plasma, wherein the ambient temperature is near room temperature, is preferred.

Cold gas plasma is a vacuum process. Typically, plasma is composed of highly excited atomic, molecular, ionic, and radical species. Although the electron temperature in plasma can be as high as 5000° K., the bulk temperature of the gas is essentially ambient because of the vacuum conditions.

Plasma treatment may carried out to introduce functional groups containing atoms such as oxygen or nitrogen onto the surface of materials. A polymeric or elastomeric material is placed in a vessel containing an inert gas or a non-carbon-containing gas such as argon, neon, helium, nitrogen, ammonia, nitrous oxide, oxygen, or air, and it is exposed to a plasma generated by a plasma (glow) discharge. For example, the surface of polyethylene normally consists solely of carbon and hydrogen. However, in an appropriate plasma, the surface becomes activated so as to contain one or more kinds of functional groups, including, but not limited to, hydroxyl, carbonyl, peroxyl, carboxyl, azido, amino, and substituted amino groups.

Suitable methods for producing plasma discharge include direct current discharge, radio-wave discharge, and microwave discharge. It is believed that free radicals are generated on the surface of the polymeric material by the action of the plasma. Subsequently, the radicals are exposed to air and reacted with oxygen to form functional groups on the surface of the polymeric material. Alternatively, plasma treatment under a low pressure of nitrogen, oxygen, or air can produce functional groups directly on the polymeric material.

For example, functional group-grafted polyurethane membranes may be prepared according to the procedure of Ozdemir Y. et al. 2002 J Mater Sci Mater Med 13, 1147-51. The polyurethane membranes were modified on the surfaces thereof with hydroperoxide groups via oxygen plasma discharge treatment. Following surface activation, the hydroperoxide groups were graft-polymerized with 1-acryloyl benzotriazole (AB) in the presence of N,N-dimethylaniline. The grafted AB groups may be substituted by carboxyl groups via a substitution reaction with sodium hydroxide or may be substituted by primary amino groups via a substitution reaction with ethylene diamine. The carboxyl or primary amino groups may then be coupled with heparin using a water-soluble carbodiimide.

Measurement of the water contact angle, chemical analysis via electron spectroscopy, and attenuated total reflection Fourier-transform infrared spectroscopy may be used to characterize the modified surfaces. AB grafting decreases the water contact angle of the polyurethane. Introduction of functional groups, such as carboxyl and primary amino, and heparin immobilization decreases the water contact angle further, which is indicative of increased hydrophilicity of the modified surfaces.

The amount of heparin immobilized covalently may be determined by the toluidine blue method. The immobilized heparin is stable in physiological solution; release of heparin from the immobilized surfaces does not commence for at least 100 hours.

Solvent Treatment

In order to make the activation treatment more effective, treatment with a solvent is preferably carried out before the activation treatment. Solvent treatment includes immersing the polymeric material in a solvent in which the polymer is virtually insoluble under conditions that do not result in dissolution. Typically, a polymeric material is immersed in such a solvent for about 1 minute to 60 minutes at a temperature range of room temperature to about 60° C. The weight of the treated polymeric material increases by 0.2 to 10% vis-à-vis untreated material without any deformation. The treatment process is completed by drying the material quickly after removal from the solvent.

Once functional groups are introduced onto the surface of a fully cured polymer, the functional groups may be linked to molecules that are capable of interacting with biological systems or that are capable of being crosslinked in the presence of chemical crosslinking agents. Suitable molecules that can be linked to the introduced functional groups include cell-binding peptides, growth factors, collagen, gelatin, glycosaminoglycans, and the like. Development of biomaterials with biomimetic surfaces increase the likelihood of cell survival. Short peptides are flexible, experience minimal steric effects, and have low immunogenic activity. They can be synthesized easily and purified at low cost.

The most commonly used cell-binding peptides for polymer surface modification are short cell-binding peptides, such as RGD, REDV, TPGPQGIAGQRGVV (P15), and YIGSR. The conventional one-letter amino acid symbols have been used in the above sequences. The complete list of symbols and the corresponding amino acids are set forth below:
A Alanine
R Arginine
N Asparagine
D Aspartic acid
C Cysteine
Q Glutamine
E Glutamic acid
G Glycine
H Histidine
I Isoleucine
L Leucine
K Lysine
M Methionine
F Phenylalanine
P Proline
S Serine
T Threonine
w Tryptophan
Y Tyrosine
V Valine

RGD is present in fibronectin, collagen, and vitronectin; REDV is present in fibronectin, TPGPQGIAGQRGVV (P15) is present in collagen; and YIGSR is present in laminin. These short peptides are derived from native extracellular matrix (ECM) proteins. They have the ability to promote cell adhesion and cell proliferation through the targeting of specific cell membrane receptors, such as integrins. For example, RGD can be linked to hydroxyl groups, created on a polymeric surface by activation (e.g., O₂ plasma glow technology), with PMPI (N-(p-maleimidophenyl) isocyanate). The isocyanate end of PMPI reacts with the hydroxyl groups to form urethane (carbamate) linkages, and the maleimide end of PMPI reacts with the sulfhydryl groups of cysteine in proteins and peptides to attach the RGD. Optionally, linker moieties may be used to increase the space between the active RGD protein and the polymer surface. The use of linkers results in a three-dimensional coating rather than a two-dimensional coating. Three-dimensional coatings have higher receptor densities than two-dimensional coatings. Mixed polyethylene glycols of different molecular weights, for example, may be used as linkers.

Many cells adhere to the extra cellular matrix (ECM) via integrins.

Certain cells undergo apoptosis induced by inadequate cell-ECM interaction, and cell adhesion via integrin molecules is essential for cell survival. Fibronectin, one of the major constituents of ECM and an important ligand for integrin, exists abundantly in synovial fluids and tissues.

In addition to attachment of RGD to the polyurethane surface, one or more growth factors and/or small molecules that enhance cell binding, development, and cell survival or that enhance the molecular regulation of cell survival may also be attached to the surface. The interactions between cells and the extracellular cell matrices play a vital role in cell development, and can therefore enhance cell survival. Growth factors are a complex family of polypeptide hormones that are produced by the body to control growth, division, and maturation of blood cells by the bone marrow. They regulate the division and proliferation of cells and influence the growth rate of some cancers. Growth factors occur naturally, but some can be synthesized using molecular biology techniques. They are used clinically to stimulate normal white cell production following chemotherapy or bone marrow transplantation.

Addition of one or more growth factors enhances further cell development and supports regeneration of tissues, especially skeletal tissues such as spinal discs. The treated surface may then be coated with a clear film of a gel, such as collagen or gelatin, that acts as a controlled release agent as the gel hydrates. The gel may optionally contain cell-growth supporting supplements, such as vitamin C or vitamin E, which support the growth of cells surrounding the spinal discs. The thus-modified surface optionally may be coated with an additional biodegradable material (forming a sandwich structure), such as a bio-degradable polymer, for example, PLGA, to fill any voids and to control release of any bioactive agents attached to the polyurethane surface. See FIG. 9. The choice of using a sandwich structure or a simple structure will depend upon the length of time needed for regeneration and nutrient support.

Small molecules suitable for attachment to the polyurethane surface include drugs, such as anti-inflammatory agents. Inflammation plays a significant role in the apthogenesis of several spinal disorders. Ankylosing spondylitits is a chronic inflammatory arthropathy of the spine. Rheumatoid arthritis, while affecting predominately limb joints, also affects the cervical spine in a significant proportion of people. Inflammation is also involved in disorders such as disc herniation and sciatica, which have previously been thought of as being primarily mechanical or degenerative. As the inflammatory cascade and immunopathology of these conditions continue to be elucidated, it has become apparent that individual molecules may be potential targets for inactivation or down-regulation. Candidates include proinflammatory cytokines, such as TNF-alpha, cytokines, e.g., IL-1, IL-15, or enzymes enhancing the inflammation pathway, such as the cyclooxygenases. (Roberts S et. al. 2005 Current Drug Targets-Inflammation and Allergy 4, 257-266). Therefore, suitable anti-inflammatory agents include those which inactivate or down-regulate such target molecules.

Another suitable small molecule is chitosan, which can function both as a scaffold and as a drug. Chitosan is an amino-polysaccharide obtained by the alkaline deacetylation of chitin derived from crustacean shells. Chitosan/glycerophosphate may be prepared as thermosensitive solution, which is a gel at 37° C. In addition, chitosan may be prepared cross-linked with a naturally occurring cross-linking reagent, genipin, which has been used in herbal medicine and in the production of food dyes. Chitosan-genipin is useful for nucleus supplementation for a number of reasons: (1) chitosan hydrogels are neither cytotoxic nor exothermic and have excellent biocompatibility; (2) chitosan can be maintained in solution below room temperature for encapsulating living cells and therapeutic proteins, but forms a gel at a room temperature for encapsulating living cells and therapeutic proteins; (3) chondrocytes embedded in chitosan hydrogels proliferate and maintain their phenotype; (4) chitosan can be cross-linked in situ with genipin; (5) chitosan can be implanted by injection without major surgical disruption of the annulus; (6) chitosan gel permits the accumulation of an appropriate extracellular matrix, and retains more than 80% of the proteoglycan produced by entrapped nucleus cells. (Mwale et al. 2005 Tissue Engineering 11, 130).

For cell culturing use, all steps should be conducted under sterile conditions. A fully cured polyurethane foam should be sterilized first with ethylene oxide and then surface-modified with e.g., O₂ plasma glow technology to introduce hydroxyl groups. The foam is then soaked in dimethyl sulfoxide, which is sterile, and reacted with PMPI. The sulfhydryl groups of the cysteine in RGDC peptides react with the maleimide end of PMPI to attach the RGD, and hydroxyl groups on the surface-modified foam react with the isocyanate end of PMPI to form urethane (carbamate) linkages, as discussed above. The resulting foam may optionally be coated with a glaze of melted or dissolved polymer, such as PLGA, and allowed to harden. Excess glaze is removed by washing the hardened material with phosphate buffered saline (PBS).

The use of short cell-binding peptides for surface modification of polymeric implants is preferred over the use of long-chain native ECM proteins. Native ECM proteins tend to be folded randomly upon adsorption onto the surface of the implant, such that the adhesion domains are not always available sterically.

With short peptides, the useful biological activity of the adhesion domains on the surface of the substrate is usually retained. Short peptides are also flexible and experience minimal steric effect. They can be synthesized easily and can be purified at relatively low cost. They are more stable than large ECM proteins during the surface modification and sterilization processes. Short peptides also have lower immunogenic activity.

The classes of growth factors include survival-inducing factors, differentiation factors, and inflammation-inducing factors. Examples of survival-inducing growth factors include epidermal growth factor (EGF), fibroblast growth factor (FGF), platelet-derived growth factor (PDGF), and insulin-like growth factors (IGF-1 and IGF-2). An example of a differentiation growth factor is vascular epithelial growth factor (VEGF). Examples of inflammation-inducing factors include interleukin-1 (IL-1) and tumor necrosis factor α (TNFα).

The healthy human intervertebral disc contains a small cell population, even smaller than the chondrocyte density seen in articular cartilage; with aging and degeneration, this cell population decreases even further. Apoptosis, programmed cell death, may be an important event that contributes to the death of cells in the disc. Apoptosis is an important type of cell death that plays a role in development, tissue homeostasis, and in numerous diseases. Cytokines, insulin-like growth factor-1 (IGF-1) and platelet-derived growth factor (PDGF) are effective in decreasing apoptosis in vitro. Selected cytokines can retard or prevent programmed cell death. Gruber et al. 2000 Spine 25, 2153-2157).

Cell-adhesive RGD-containing peptides may be grafted to a carboxylated polyurethane copolymer backbone according to the one-step or two-step method of Lin HB et al. 1994 J Biomed Mater Res 28, 329-42. In the one-step method, a free peptide is coupled directly onto a carboxylated polyurethane via amide linkage formation. The coupling reaction is performed under dry nitrogen at room temperature in dimethyl formamide solution, with (3-dimethylaminopropyl)3-ethylcarbodiimide hydrochloride (EDCI) as a coupling reagent. In the two-step method, first a protected peptide is coupled onto a carboxylated polyurethane as in the one-step method. In the second step, the protected groups of the grafted peptide are cleaved off.

In vitro endothelial cell adhesion experiments by Lin et al. showed that without the presence of serum in the culture medium, GRGDSY- and GRGDVY-grafted polyurethanes enhanced cell attachment and spreading dramatically compared with the starting, carboxylated, and GRGESY-grafted polymers. Increasing the peptide density from 100 to 250 pmol/g polymer for the GRGDSY- and GRGDVY-grafted polyurethanes resulted in an increase in cell attachment. With approximately the same peptide density (100 or 250 pmol/g polymer), the GRGDVY-grafted polymers supported more adherent cells than did the GRGDSY-grafted polymers

Similar trends were observed in in vitro endothelial cell growth studies using culture medium containing serum and endothelial cell growth supplement. The GRGDSY- and GRGDVY-grafted polyurethanes promoted more cell growth than did the starting polyurethane. However, the presence of adhesive serum proteins and growth factor diminished the differences between the cell-adhesive peptide grafted polymers and the GRGESY-grafted polymers.

Collagens for use in the present invention may be in the fibrillar or nonfibrillar form. Fibrillar collagens are generally preferred for tissue augmentation applications due to their increased persistence in vivo. Nonfibrillar collagens, including chemically modified collagens such as succinylated or methylated collagen, may be preferable in certain situations. Succinylated and methylated collagens can be prepared according to the methods described in U.S. Pat. No. 4,164,559 (which is hereby incorporated by reference in its entirety). Noncrosslinked collagens for use in the present invention are normally in aqueous suspension at a concentration between about 20 mg/ml to about 120 mg/ml, preferably, between about 30 mg/ml to about 80 mg/ml. Fibrillar collagen in suspension at various collagen concentrations is commercially available.

In general, collagen and gelatin from any source may be used in the practice of the present invention; for example, collagen may be extracted and purified from human or other mammalian source, or may be recombinantly or otherwise produced. Collagen of any type, including, but not limited to, types I, II, III, IV, or any combination thereof, may be used, although type I is generally preferred. Either atelopeptide or telopeptide-containing collagen may be used; however, when collagen from a xenogeneic source, such as bovine collagen, is used, atelopeptide collagen is generally preferred, because of its reduced immunogenicity compared to telopeptide-containing collagen. The collagen should be in a pharmaceutically pure form such that it can be incorporated into a human body without generating any significant immune response.

Collagen in its native state contains lysine residues having primary amino groups capable of covalently binding with chemical crosslinking agents, and therefore need not be chemically modified in any way prior to reaction with the desired crosslinking agent. Although intact collagen is preferred, denatured collagen, commonly known as gelatin, can also be used in the present invention.

Glycosaminoglycans for use in the present invention include, without limitation, hyaluronic acid, chondroitin sulfate A, chondroitin sulfate C, dermatan sulfate, keratan sulfate, keratosulfate, chitin, chitosan, heparin, and derivatives or mixtures thereof. For example, heparin may be coupled with primary amino or carboxyl groups on an activated polymer surface using water-soluble carbodiimide (Kang I K et al. 1996 Biomaterials 17(8), 841-7). Depending on the nature of the crosslinking agent, the glycosaminoglycans may need to be modified, such as by deacetylation or desulfation, in order to provide groups capable of binding with the crosslinking agent. In general, glycosaminoglycans can be deacetylated, desulfated, or both, as applicable, by the addition of a strong base, such as sodium hydroxide, to the glycosaminoglycan. Deacetylation and/or desulfation provides primary amino groups on the glycosaminoglycan which are capable of covalently binding with hydrophobic or hydrophilic crosslinking agents.

Mixtures of various species of glycosaminoglycan, various types of collagen, and various types of gelatin, or mixtures thereof may be used in the present invention.

Crosslinking Agents

When collagen and/or glycosaminoglycans are used in the present invention, they may be crosslinked with any chemical crosslinking agent that is capable of covalently binding these biomaterials so as to form a crosslinked biomaterial network. Functionally activated polyethylene glycols, glutaraldehyde, diphenylphosphoryl azide are known crosslinking agents. Care should be taken with glutaraldehyde because it may be cytotoxic. Other crosslinking agents include various hydrophobic polymers containing two or more succinimidyl groups, such as disuccinimidyl suberate, bis(sulfosuccinimidyl) suberate, or dithiobis(succinimidyl-propionate). In addition, polyacids can be derivatized to contain two or more succinimidyl groups and, in the derivatized form, can be used to crosslink collagen and glycosaminoglycans. A mixture of hydrophobic and hydrophilic crosslinking agents can also be used. See U.S. Pat. No. 6,962,979.

Synthetic hydrophilic polymers, such as functionally activated polyethylene glycols, are examples of hydrophilic crosslinking agents. Various activated forms of polyethylene glycol are described in detail in U.S. Pat. No. 5,328,955. Synthetic hydrophilic polymers may be multifunctionally activated, e.g. difunctionally activated. Difunctionally activated forms of PEG include succinimidyl glutarate (SG-PEG), PEG succinimidyl (SE-PEG), PEG succinimidyl succinamide (SSA-PEG), and PEG succinimidyl carbonate (SC-PEG).

Surface Modification Via Chemical Treatment

In another aspect of the present invention, the polymer surface may be modified by exposure to a chemical that forms linkers on the surface. The linkers may then be chemically and covalently attached to additional materials so as to produce a polymer surface coated with the additional material. As with the activation treatment processes described above, chemical surface treatment is a fast and efficient method for improving the adhesion properties and other surface characteristics of a variety of polymeric materials.

One such example of chemical treatment is the pre-impregnation of a segmented polyurethane (SPU) film with camphorquinone, as described in Magoshi T. and Matsuda T. 2002 Biomacromolecules 3(5), 976-83. Acrylic acid was then graft-polymerized onto the SPU film using visible light irradiation. Next, multiply styrenated albumin, styrenated heparin, or a mixture thereof was adsorbed onto the grafted surface, followed by visible light irradiation in the presence of carboxylated camphorquinone. Finally, the polyacrylic acid graft and the heparin/albumin were crosslinked, and the heparin/albumin were crosslinked to one another, so as to form covalent bonds and to enforce the formation of a stable immobilized layer. At each step the surfaces formed were analyzed with X-ray photoelectron spectroscopy and Fourier transform-infrared spectroscopy. Confocal laser scanning microscopy was used to determine the thickness of the heparin/albumin layer.

Platelet adhesion is markedly reduced on these polymerized albuminated, polymerized heparinized, and mixed polymerized heparin/albumin surfaces. Adhesive and proliferative potentials of endothelial cells are comparable to those of commercial tissue culture dishes. Co-immobilization of fibronectin and basic fibroblast growth factor enhances these potentials.

In another example of chemical treatment, collagen or RGD may be bound to a polymer surface via an azide-ester linkage. The polymer surface is first reacted with 5-azido-2-nitrobenzoyloxy-N-hydroxysuccinimide in the presence of UV light, thereby binding the azido group to the polymer surface. Collagen or RGD are then linked to the succinimide moiety via an ester linkage. The resulting modified polymer surface exhibits enhanced cell adhesion and spreading.

Other Materials

Anorganic bone matrix (ABM), a bone graft material utilized routinely, when activated by the cell binding peptide P-15 (15 amino acids, not containing RGD) produced larger, more spread cells compared with smaller cells with apoptotic cellular blebs on unactivated ABM. Anchorage-dependent human foreskin fibroblasts osteogenic MC3T3-E1 cells were seeded on ABM or ABM/P-15 and compared for cell viability and apoptosis. After serum withdrawal, viability and apoptosis level were significantly (p<0.05) improved for cells on ABM/P-15 compared to cells on ABM. In addition, viable cell attachment was significantly greater on cells cultured on ABM/P-15 compared with demineralized freeze-dried bone allograft. Hanks T. et al. 2004 Biomaterials 25(19), 4831-6.

### EXAMPLE

The following is an example of some of the steps and materials that may be employed in the method of the present invention:

The herniated portion of one or more spinal discs is removed surgically. A delivery tool is used to deliver a compressed, surface treated, cured, biodegradable polymer, e.g. medical grade polyurethane foam, to the defective portion of the disc. A calculated and pre-selected amount of compressed polymer is delivered to fill the void in the disc when the polymer expands. The tool is used to cut away the delivered polymer from the undelivered polymer, and the tool is then withdrawn. The polymer expands when it is released from the delivery tool and fills the void in the disc. A mechanical closure device or tissue glue is implanted to seal the opening in the annulus.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

Where a range of values is provided herein, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

It must be noted that as used herein and in the appended claims, reference to a singular item, includes the possibility that there are plural of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless defmed otherwise, all technical and scientific terms used herein have the same meaning as commonly understood by one of ordinary skill in the art to which this invention belongs.

All publications mentioned herein are incorporated herein by reference to disclose and describe the methods and/or materials in connection with which the publications are cited. It is understood that the present disclosure supercedes any disclosure of an incorporated publication to the extent there is a contradiction.

That which is claimed is:

1. A method of treating a herniated spinal disc, the method comprising:
providing a material comprising a cured polymer, wherein the polymer is provided in a first stage;
delivering a selected amount of the material in the first stage into a defective herniated region of a spinal disc; and
transitioning the material from the first stage to a second stage, wherein the material in the second stage fills the void caused by a herniation and provides mechanical and material characteristics which mimic substantially that of the natural spinal disc and supports cell regeneration and restoration.

2. The method of claim 1, wherein the first stage is a flowable and the second stage is not flowable.

3. The method of claim 2, wherein the first stage is a fluid and the second stage is a monolithic structure.

4. The method of claim 2, wherein the first stage comprises a plurality of smaller units and the second stage is a larger monolithic structure.

5. The method of claim 4, wherein the plurality of smaller units comprises microgel particles.

6. The method of claim 1, wherein the step of transitioning is active.

7. The method of claim 6, wherein the active transition comprises applying energy or a chemical to the implanted material.

8. The method of claim 1, wherein the step of transitioning is passive.

9. The method of claim 8, wherein the passive transition comprises allowing the implanted material to swell or expand.

10. The method of claim 9, wherein the swelling or expansion is caused by body fluids within the disc system and by body temperature, and the swelling or expansion is controlled.

11. The method of claim 9, wherein the swelling is caused by fluid absorption.

12. The method of claim 1, wherein the step of providing the material in the first stage comprises compressing the material to a reduced size and the step of transitioning the implanted material to the second stage comprises expanding the material to a larger size.

13. The method of claim 12, wherein the material is provided in the compressed stage within a delivery tool and the material transitions to the expanded stage upon expulsion from the delivery tool.

14. The method of claim 12, wherein the material is provided in the compressed stage in a biodegradable casing and the material achieves the expanded stage upon degradation of the casing.

15. The method of claim 12, wherein the material is foam.

16. The method of claim 15, wherein the material comprises a plurality of foam units.

17. The method of claim 15, wherein the surface of the foam is activated to introduce functional groups thereon.

18. The method of claim 17, wherein the functional groups are linked to molecules that are capable of interacting with biological systems or that are capable of being crosslinked in the presence of chemical crosslinking agents.

19. The method of claim 15, wherein the surface of the foam is chemically treated, such that the foam may be chemically and covalently linked to an additional material, which coats the foam.

20. The method of claim 1, wherein the material is selected from a hydrogel, a microgel particle, a foam, a cord and a bead.

21. The method of claim 20, wherein the surface of the material is activated to introduce functional groups thereon.

22. The method of claim 21, wherein the functional groups are linked to molecules that are capable of interacting with biological systems or that are capable of being crosslinked in the presence of chemical crosslinking agents.

23. The method of claim 20, wherein the surface of the material is chemically treated, such that the material may be chemically and covalently linked to an additional material, which coats the material.

24. The method of claim 1, wherein the polymer is polyurethane.

25. The method of claim 1, wherein providing the material in the first stage comprises placing at least a portion of the material within a biodegradable casing.

26. The method of claim 1, wherein providing the material in the first stage comprises placing the material within a delivery tool.

27. The method of claim 1, wherein the disc is augmented by implantation of one or more closure devices.

28. The method of claim 27, wherein the surface of the closure devices is treated with cell adhesion molecules or anti-cell adhesion molecules to enhance cell proliferation, cell differentiation, and protein synthesis of disc-related cell types.

29. A method of treating a herniated spinal disc, the method comprising:
providing a material comprising a cured, surface treated, biodegradable, polyurethane foam, wherein the material is provided in a first stage;
delivering a selected amount of the material in the first stage into a defective herniated region of a spinal disc; and
transitioning the material from the first stage to a second stage, wherein the material in the second stage fills the void caused by a herniation and provides mechanical and material characteristics which mimic substantially that of the natural spinal disc and supports cell regeneration and restoration.
